(19) United States
(12) Patent Application Publication
(10) Pub. No.: US 2021/0038270 A1
(43) Pub. Date: Feb. 11, 2021

(54) Ankle (Tibio-Talar) Fusion Nail

(71) Applicant: Texas Tech University System, Lubbock, TX (US)

(72) Inventor: Amr Abdelgawad, El Paso, TX (US)

(21) Appl. No.: 16/978,836

(22) PCT Filed: Mar. 8, 2019
(86) PCT No.: PCT/US2019/021339

§ 371 Date: Sep. 8, 2020

Related U.S. Application Data
(60) Provisional application No. 62/640,604, filed on Mar. 9, 2018.

(51) Int. Cl. A61B 17/72

(52) U.S. Cl. CPC A61B 17/7291; A61B 17/725; A61B 17/7241

(57) ABSTRACT

The present invention includes a tibio-talar device for providing stabilizing support between a tibia and a talus including: a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has openings are positioned along the length of the bone nail, and wherein the screw engages the bone nail to lock its rotation in relation to the tibia and talus and optionally for compression of two or more bones selected from tibia to talus, fibula to tibia, or fibula to talus,

### CROSS-REFERENCE TO RELATED APPLICATIONS

None.

### TECHNICAL FIELD OF THE INVENTION

The present invention relates in general to the field of arthrodesis, and more particularly, to a novel ankle (tibio-talar) fusion nail.

### STATEMENT OF FEDERALLY FUNDED RESEARCH

None.

### BACKGROUND OF THE INVENTION

Without limiting the scope of the invention, its background is described in connection with Joint arthrodesis and arthroplasty.

Ankle fusion is currently done by screws with or without plates or external fixators. Both of these have their disadvantages. The nail system when used for nail fixation requires fixation of the subtalar joint. Thus, when a nail is used to fuse an ankle (tibio-talar) joint, it will have to also fuse another joint (subtalar or the talo-calcaneal joint). This means that the ankle joint cannot be fused with a nail unless another joint is fused with it.

U.S. Pat. No. 8,585,744, issued to Duggal, et al., for a Joint arthrodesis and arthroplasty. Briefly, these inventors teach an implantable fixation system for fusing a joint between a first bone and a second bone. The system may include an anchor, standoff, bolt, and cortical washer. The system may be implanted across the joint along a single trajectory, the length of the system adjustable to provide compressive force between the anchor and the cortical washer. The system may be implanted across a tibio-talar joint with the anchor positioned in the sinus tarsi. A spacing member may be inserted between the two bones and the fixation system implanted to extend through an opening in the spacing member. The spacing member may be anatomically shaped and/or provide deformity correction. An ankle arthroplasty system may include a tibial plate, a talar plate, and a bearing insert. The plates may be anchored to the tibia and talus along a single trajectory. The ankle arthroplasty system may be revisable to a fusion system.

U.S. Pat. No. 9,125,695, issued to Early et al., “Ankle fusion nail apparatus and method” claims to teach an ankle fusion nail apparatus and method that includes a first, tibial component that includes a hole there through. The tibial component may include, among other things, a base. A second, talar component may include a hole there through, also, and, among other things, a base and a top. The talar component may be separate from the talar component. A third, central component may be provided that may be separate from the first tibial component and the second talar component. The central component may be conformed to connect with the tibial base and the talar top such that the central component joins the tibial and talar components together and aligns them as the central component is connected with the tibial base and the talar top. The present invention is advantageous over the claimed teaching of this published application in that (1) the straight portion of an embodiment of the present invention can be longer and can extend to the tibial shaft; (2) the straight portion of an embodiment of the present invention requires less bone reaming, permitting insertion on the talus or the tibia, whereas a curved nail requires more bone reaming with the attendant major bone loss and possible fracturing of the talus ; (3) an embodiment of the present invention may be inserted laterally and not medially, whereas medial insertion is difficult and risks damage to the posterior tibial nerve, the posterior tibial artery, and the tibialis posterior tendon; (4) locking screws of the present invention make the present invention very stable and should help with quicker union; and (5) an embodiment of the present invention requires less talar bone loss for insertion and better mechanical stability than the claimed teaching of the issued patent.

### SUMMARY OF THE INVENTION

In one embodiment, the present invention is a tibio-talar device for providing stabilizing support between a tibia and a talus comprising: a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a screw, wherein the openings are positioned along the length of the bone nail, and wherein the screw engages the bone nail to lock its rotation in relation to the tibia and talus and optionally for compression of two or more bones selected from tibia to talus, fibula to tibia, or fibula to talus. In one aspect, the device further comprises one or more screws, wherein at least of the one or more screws is adapted to engage one of the openings in the bone nail. In another aspect, the bone nail is at least one of: further affixed to the tibia and talus with a biocompatible adhesive; contoured to lock proximally in the tibia from lateral to medial; provided in increments of approximately 5 cm; or is at least one of titanium, stainless steel, nitinol or other biocompatible material. In another aspect, the device further comprises one or more openings to receive screws that pass from the tibia or the fibula through the bone nail to the talus. In another aspect, the bone nail has an upper portion, a middle portion, and a lower portion, wherein the upper portion and the lower portion have the same curvature, the upper portion is straight and the lower portion is curved, wherein the lower and upper portions are curved but have different curvatures, or wherein the upper portion is curved, the middle portion is curved, and the lower portion is straight. In another aspect, at least one of the openings is threaded to engage the at least one of the one or more screws to compress the fibula to both the tibia and talus to enhance fusion. In another aspect, at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded. In another aspect, at least one of the screws is adapted to engage bone tissue with a threaded portion to enhance compression and stability of fusion. In another aspect, at least one of the screws is adapted to lock into the bone nail. In another aspect, the openings have a longitudinal axis from lateral to medial, and are horizontal.

In another embodiment, the present invention is an ankle arthrodesis nail kit comprising: a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a locking screw using a surface or a locking mechanism, wherein the openings are positioned along the length of the bone nail, and wherein the screw engages the bone nail to lock its rotation in relation to the tibia and talus and optionally for compression of two or more bones selected from tibia to talus, fibula to tibia, or fibula to talus. In another aspect, the kit further comprises one or more screws, wherein for each of the one or more screws, at least a portion of an end proximate to a head of the screw is not threaded and at least a portion of a distal end is threaded for engagement with the bone nail, and wherein each of the one or more screws is adapted to lock into one of the openings in the bone nail. In another aspect, the bone nail is at least one of: further affixed to the tibia and talus with a biocompatible adhesive; contoured to lock proximally in the tibia from lateral to medial; provided in increments of approximately 5 cm; or is at least one of titanium, stainless steel, nitinol or other biocompatible material. In another aspect, the nail further comprises one or more openings to receive screws that pass from the tibia or the fibula through the bone nail to the talus. In another aspect, the nail bone has an upper portion, a middle portion, and a lower portion, wherein the upper portion and the lower portion have the same curvature, the upper portion is straight and the lower portion is curved, wherein the lower and upper portions are curved but have different curvatures, or wherein the upper portion is curved, the middle portion is curved, and the lower portion is straight. In another aspect, at least one of the openings is threaded to engage at least one of the one or more screws to compress the fibula to both the tibia and talus to enhance fusion. In another aspect, at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded. In another aspect, at least one of the screws is adapted to engage bone tissue with a threaded portion to enhance compression and stability of fusion. In another aspect, at least one of the screws is adapted to lock into the bone nail. In another aspect, the openings have a longitudinal axis from lateral to medial, and are horizontal.

In another embodiment, the present invention includes a kit for use in a method for conducting an ankle arthrodesis system for providing stabilizing support between a tibia and a talus, the method comprising: identifying a patient in need of an ankle arthrodesis; inserting a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a screw, wherein the openings are positioned along the length of the bone nail, and wherein the screw engages the bone nail to lock its rotation in relation to the tibia and talus and optionally for compression of two or more bones selected from tibia to talus, fibula to tibia, or fibula to talus; anchoring the bone nail without obstructing the subtalar joint; and inserting one or more screws through one or more of the openings, wherein for each of the one or more screws, at least a portion of an end proximate to a head of the screw is not threaded and at least a portion of a distal end is threaded for engagement with the bone nail. In one aspect, the opening is defined further as being adapted for the bone anchor to at least one of traverse from talus to tibia or traverse from tibia to talus. In another aspect, the bone nail is at least one of: further affixed to the tibia and talus with a biocompatible adhesive; contoured to lock proximally in the tibia from lateral to medial; provided in increments of approximately 5 cm; or is at least one of titanium, stainless steel, nitinol or other biocompatible material. In another aspect, the bone nail comprises one or more openings to receive screws that pass from the tibia or the fibula through the bone nail to the talus. In another aspect, the bone nail has an upper portion, a middle portion, and a lower portion, wherein the upper portion and the lower portion have the same curvature, the upper portion is straight and the lower portion is curved, wherein the lower and upper portions are curved but have different curvatures, or wherein the upper portion is curved, the middle portion is curved, and the lower portion is straight. In another aspect, the bone nail has 2, 3, 4, 5, 6, or 8 openings that each are capable of supporting a screw, wherein the openings are positioned along the length of the bone nail, and wherein optionally one of the openings permits affixing the bone screw to the talus. In another aspect, at least one of the openings is threaded to engage the one or more screws to compress the fibula to both the tibia and talus to enhance fusion. In another aspect, at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded. In another aspect, at least one of the screws is adapted to engage bone tissue with a threaded portion to enhance compression and stability of fusion. In another aspect, at least one of the screws is adapted to lock into the bone nail. In another aspect, the openings have a longitudinal axis from lateral to medial, and are horizontal.

In another embodiment, the present invention includes a tibio-talar device for providing stabilizing support between a tibia and a talus comprising: a bone nail adapted to traverse the tibia and the talus, wherein the bone nail is curved such that it traverses the tibia and enters the talus at an angle, wherein the bone nail is angled from lateral to medial and anterior to posterior, wherein the bone nail is configured to provide for intra medullary fusion of the ankle without obstructing the subtalar joint, wherein the bone nail has 2, 3, 4, 5, 6, 7, or 8 openings that each are capable of engaging a locking screw using a surface or a locking mechanism, wherein the openings are positioned along the length of the bone nail, and wherein the screw engages the bone nail to lock its rotation in relation to the tibia and talus and optionally for compression of two or more bones selected from tibia to talus, fibula to tibia, or fibula to talus; and one or more screws, wherein for each of the one or more screws, at least a portion of an end proximate to a head of the screw is not threaded and at least a portion of a distal end is threaded for engagement with the bone nail, and wherein each of the one or more screws is adapted to lock into one of the openings in the bone nail.

In one embodiment, the present invention includes a tibio-talar device for providing stabilizing support between a tibia and a talus including: a bone nail adapted to traverse the tibia and the talus, wherein the bone nail is curved such that it traverses the tibia and enters the talus at an angle, wherein the nail is angled from lateral to medial and anterior to posterior, wherein the bone nail is configured to provide for intra medullary fusion of the ankle without obstructing the subtalar joint. In one aspect, the nail is further affixed to the tibia and talus with a biocompatible adhesive. In another aspect, the nail is contoured to lock proximally in the tibia from lateral to medial. In another aspect, the bone nail is provided in increments of approximately 5 cm. In another aspect, the bone nail is at least one of titanium or stainless steel. In another aspect, the bone nail has an upper and a lower portion, wherein the upper portion and the lower portion have the same curvature, the upper portion is straight and the lower portion is curved, or wherein the lower and upper portion are curved but have a radius of curvature. In another aspect, the bone nail has 2, 3, 4, 5, 6, or 8 openings that each are capable of supporting a screw, wherein the holes are positioned along the length of the bone nail, and wherein optionally one of the holes permits affixing the bone screw to the talus.

In another embodiment, the present invention includes an ankle arthrodesis nail including: a bone nail adapted to traverse the tibia and the talus, wherein the bone nail is curved such that it traverses the tibia and enters the talus at an angle, wherein the nail is angled from lateral to medial, and wherein the bone nail is configured to provide for intra medullary fusion of the ankle, wherein the nail locks proximally in the tibia from lateral to medial, without obstructing the subtalar joint. In one aspect, the nail is further affixed to the tibia and talus with a biocompatible adhesive. In another aspect, the nail is contoured to lock proximally in the tibia from lateral to medial. In another aspect, the bone nail is provided in increments of approximately 5 cm. In another aspect, the bone nail is at least one of titanium or stainless steel. In another aspect, device further includes one or more opening to receive screws that pass from the tibia or the fibula through the nail to the talus. In another aspect, the bone nail has an upper portion, a middle portion, and a lower portion, wherein the upper portion and the lower portion have the same curvature, the upper portion is straight and the lower portion is curved, wherein the lower and upper portion are curved but have different radii of curvature, or wherein the upper portion is curved, the middle portion is curved, and the lower portion is straight. In another aspect, the bone nail has 2, 3, 4, 5, 6, or 8 openings that each are capable of supporting a screw, wherein the holes are positioned along the length of the bone nail, and wherein optionally one of the holes permits affixing the bone screw to the talus. The holes can be threaded to allow the locking screws to engage into the holes to compress the fibula to both the tibia and the talus to enhance the fusion.

In yet another embodiment, the present invention includes a method for conducting an ankle arthrodesis system for providing stabilizing support between a tibia and a talus, the method including: identifying a patient in need of an ankle arthrodesis; inserting a bone nail adapted to traverse the tibia and the talus, wherein the bone nail is curved such that it traverses the tibia and enters the talus at an angle, wherein the nail is angled from lateral to medial , wherein the bone nail is configured to provide for intra medullary fusion of the ankle; and anchoring the bone nail without obstructing the subtalar joint. In one aspect, the opening is defined further as being adapted for the bone anchor to at least one of traverse from talus to tibia or traverse from tibia to talus. In another aspect, the nail is further affixed to the tibia and talus with a biocompatible adhesive. In another aspect, the nail is contoured to lock proximally in the tibia from lateral to medial. In another aspect, the bone nail is provided in increments of approximately 5 cm. In another aspect, the bone nail is at least one of titanium or stainless steel. In another aspect, the method further including inserting one or more screws through openings in the bone nail, wherein the screws pass from the tibia or the fibula through the nail to the talus. In another aspect, the bone nail has an upper portion, a middle portion, and a lower portion, wherein the upper portion and the lower portion have the same curvature, the upper portion is straight and the lower portion is curved, wherein the lower and upper portion are curved but have different radii of curvature, or wherein the upper portion is curved, the middle portion is curved, and the lower portion is straight. In another aspect, the bone nail has 2, 3, 4, 5, 6, or 8 openings that each are capable of supporting a screw, wherein the holes are positioned along the length of the bone nail, and wherein optionally one of the holes permits affixing the bone screw to the talus. The holes can be threaded to allow the locking screws to engage into the holes to compress the fibula to both the tibia and the talus to enhance the fusion.

In yet another embodiment, the present invention includes a tibio-talar device for providing stabilizing support between a tibia and a talus including a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a screw, wherein the openings are positioned along the length of the bone nail. In one aspect, the device further includes one or more screws, wherein at least of the one or more screws is adapted to engage one of the openings in the bone nail. In another aspect, the bone nail is further affixed to the tibia and talus with a biocompatible adhesive. In another aspect, the bone nail is contoured to lock proximally in the tibia from lateral to medial. In another aspect, the bone nail is provided in increments of approximately 5 cm. In another aspect, the bone nail is at least one of titanium or stainless steel. In another aspect, the tibio-talar device further includes one or more openings to receive screws that pass from the tibia or the fibula through the bone nail to the talus. In another aspect, the bone nail has an upper and a lower portion, wherein the upper portion and the lower portion have the same curvature, the upper portion is straight and the lower portion is curved, or wherein the lower and upper portion are curved but have a radius of curvature. In another aspect, the bone nail has an upper portion, a middle portion, and a lower portion, wherein the upper portion and the lower portion have the same curvature, the upper portion is straight and the lower portion is curved, wherein the lower and upper portion are curved but have different radii of curvature, or wherein the upper portion is curved, middle portion is curved, and the lower portion is straight. In another aspect, at least one of the openings is oriented horizontally in the bone nail. In another aspect, at least one of the openings is threaded to engage the at least one of the one or more screws to compress the fibula to both the tibia and talus to enhance fusion. In another aspect, for at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded. In another aspect, at least one of the screws is adapted to engage bone tissue with a threaded portion to enhance compression and stability of fusion. In another aspect, at least one of the screws is adapted to lock into the bone nail.

In yet another embodiment, the present invention includes an ankle arthrodesis nail kit including a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a locking screw using a surface or a locking mechanism, wherein the openings are positioned along the length of the bone nail. In one aspect, the ankle arthrodesis nail kit further includes one or more screws, wherein for each of the one or more screws, at least a portion of an end proximate to a head of the screw is not threaded and at least a portion of a distal end is threaded for engagement with the bone nail, and wherein each of the one or more screws is adapted to lock into one of the openings in the bone nail. In another aspect, the bone nail is further affixed to the tibia and talus with a biocompatible adhesive. In another aspect, the bone nail is contoured to lock proximally in the tibia from lateral to medial. In another aspect, the bone nail is provided in increments of approximately 5 cm. In another aspect, the bone nail is at least one of titanium or stainless steel.

In another aspect, the ankle arthrodesis nail kit further includes one or more openings to receive screws that pass from the tibia or the fibula through the bone nail to the talus. In another aspect, the bone nail has an upper portion, a middle portion, and a lower portion, wherein the upper portion and the lower portion have the same curvature, the upper portion is straight and the lower portion is curved, wherein the lower and upper portions are curved but have different curvatures, or wherein the upper portion is curved, the middle portion is curved and the lower portion is straight. In another aspect, at least one of the openings is oriented horizontally in the bone nail. In another aspect, at least one of the openings is threaded to engage at least one of the one or more screws to compress the fibula to both the tibia and talus to enhance fusion. In another aspect, for at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded. In another aspect, at least one of the screws is adapted to engage bone tissue with a threaded portion to enhance compression and stability of fusion. In another aspect, at least one of the screws is adapted to lock into the bone nail. In another aspect, the bone nail has an upper and a lower portion, wherein the upper portion and the lower portion have the same curvature, the upper portion is straight and the lower portion is curved, or wherein the lower and upper portion are curved but have a radius of curvature.

In yet another embodiment, the present invention includes a method for conducting an ankle arthrodesis system for providing stabilizing support between a tibia and a talus, the method including identifying a patient in need of an ankle arthrodesis; inserting a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a screw, wherein the openings are positioned along the length of the bone nail; anchoring the bone nail without obstructing the subtalar joint; and inserting one or more screws through one or more of the openings, wherein for each of the one or more screws, at least a portion of an end proximate to a head of the screw is not threaded and at least a portion of a distal end is threaded for engagement with the bone nail. In one aspect, opening is defined further as being adapted for the bone anchor to at least one of traverse from talus to tibia or traverse from tibia to talus. In another aspect, the bone nail is further affixed to the tibia and talus with a biocompatible adhesive. In another aspect, the bone nail is contoured to lock proximally in the tibia from lateral to medial. In another aspect, the bone nail is provided in increments of approximately 5 cm. In another aspect, the bone nail is at least one of titanium or stainless steel. In another aspect, the bone nail comprises one or more openings to receive screws that pass from the tibia or the fibula through the bone nail to the talus. In another aspect, the bone nail has an upper portion, a middle portion, and a lower portion, wherein the upper portion and the lower portion have the same curvature, the upper portion is straight and the lower portion is curved, wherein the lower and upper portions are curved but have different curvatures, or wherein the upper portion is curved, the middle portion is curved and the lower portion is straight. In another aspect, the bone nail has 2, 3, 4, 5, 6, or 8 openings that each are capable of supporting a screw, wherein the openings are positioned along the length of the bone nail, and wherein optionally one of the openings permits affixing the bone screw to the talus. In another aspect at least one of the openings is oriented horizontally in the bone nail. In another aspect, at least one of the openings is threaded to engage the one or more screws to compress the fibula to both the tibia and talus to enhance fusion. In another aspect, for at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded. In another aspect, at least one of the screws is adapted to engage bone tissue with a threaded portion to enhance compression hence stability of the fusion. In another aspect, at least one of the screws is adapted to lock into the bone nail. In another aspect, the bone nail has an upper and a lower portion, wherein the upper portion and the lower portion have the same curvature, the upper portion is straight and the lower portion is curved, or wherein the lower and upper portion are curved but have a radius of curvature.

In yet another embodiment, the present invention includes a tibio-talar device for providing stabilizing support between a tibia and a talus including a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a locking screw using a surface or a locking mechanism, wherein the openings are positioned along the length of the bone nail; and one or more screws, wherein for each of the one or more screws, at least a portion of an end proximate to a head of the screw is not threaded and at least a portion of a distal end is threaded for engagement with the bone nail, and wherein each of the one or more screws is adapted to lock into one of the openings in the bone nail. In another aspect, the bone nail has an upper and a lower portion, wherein the upper portion and the lower portion have the same curvature, the upper portion is straight and the lower portion is curved, or wherein the lower and upper portion are curved but have a radius of curvature.

### BRIEF DESCRIPTION OF THE DRAWINGS

For a more complete understanding of the features and advantages of the present invention, reference is now made to the detailed description of the invention along with the accompanying figures and in which:

FIG. 1 shows a lateral and an anterior view of the anatomy of the ankle and subtalar joint.

FIG. 2 shows a posterior view of an ankle fusion by plate of the prior art.

FIG. 3 shows an anterior view of an ankle fusion by screws of the prior art.

FIG. 4 shows an isometric view of an ankle fusion by external fixator of the prior art.

FIG. 5 shows a lateral view of an ankle fusion by nail of the prior art (which will lead to subtalar fusion).

FIG. 6 shows a lateral and a posterior view of an ankle fusion by nail of the prior art (which will lead to subtalar fusion).

FIG. 7 shows an isometric anterior view of the new device positioned between the tibia and the talus.

FIG. 8 shows a close-up lateral view of the new device positioned between the tibia and the talus (as seen from the anterior lateral view).

FIG. 9 shows an anterior view of the new device positioned between the tibia and the talus.

FIG. 10 shows a close-up lateral view of the new device positioned between the tibia and the talus.

FIG. 11 shows a coronal cut of a CT drawing showing the new device positioned between the tibia and the talus.

FIG. 12 shows a computational analysis was performed applying an axial load of 350N at the top area of the tibia simulating the weight bearing of an average person.

FIG. 13 shows a computational analysis in which the talus is subjected to high stress in the section around the nail insertion.

FIG. 14 shows a computational analysis in which the nail, tibia, and talus are shown and in which the stress is concentrated in the upper section, which carries the axial load at the first instance.

FIG. 15 shows the fixation of the nail to the bones with screws that ensures proper fixation and preventing movement during joint fusion and supporting and distributing stresses uniformly throughout the structure.

FIG. 16 shows the same image as in FIG. 15, but illustrates screw 5. Screw 5 is inserted in an inclined configuration, which is positioned from the tibia to the talus to increase fixation.

FIG. 17 shows the screw of the talus (4) and the screw which crosses from tibia to talus (5) have angle of inclination.

FIGS. 18A and 18B illustrate the angle of inclination for talus screw (4) and for tibia/talus screw (5).

FIGS. 19A and 19B illustrate one option for the nail of the present invention in which two straight segments connected each other; they have an angle of 37.24°. This nail is called “straight nail” and it is inserted from the talus to the tibia.

FIG. 19C shows that the subtalar joint is not affected when the nail is inserted and positioned. The image below shows the clearance between the nail and calcaneus or between the nail and subtalar joint.

FIGS. 20A and 20B are a lateral and front view of the nail and screw holes. FIG. 20C is an isometric view of the nail, and screws in the screw holes.

FIGS. 21A and 21B illustrate another option for the nail of the present invention in which two straight segments connected each other and shows the clearance between the nail and calcaneus or between the nail and subtalar joint, and is based on constant curved nail geometry. Only one curvature is designed and will pass through the tibia and the talus.

FIG. 21C shows the clearance between the nail trajectory and the subtalar joint is shown below; it seems to be good enough to prevent damage in the subtalar joint. In this embodiment, the nail is not as close to the calcaneus as in the figures above, however the nail is closer to the sidewall of the tibia.

FIGS. 22A and 22B are a lateral and front view of the nail and screw holes. FIG. 22C is an isometric view of the nail, and screws, in the screw holes.

FIGS. 23A and 23B illustrate another option for the nail of the present invention in which the curved nail has two different curves connected to form the nail. One curvature is designed for the tibia segment and a more pronounce curvature is selected for the talus segment.

FIG. 23C shows the clearance between the nail and subtalar joint is small but still enough to avoid damage in the calcaneus zone.

FIGS. 24A and 24B are a lateral and front view of the nail and screw holes. FIG. 24C is an isometric view of the nail, screws in the screw holes.

FIG. 25 shows an isometric anterior view of embodiment of the present invention between the tibia and the talus with locking screws.

FIGS. 26A, 26B, 26C, 26D, 26E, 26F, and 26G depict threaded openings in the bone nail, threaded screws inserted in the threaded openings, an exemplary horizontal width of the bone nail, exemplary curvatures of the bone nail, an alternative embodiment of the bone nail, an embodiment of the bone nail inserted into the tibia and the talar, and the bone removed for insertion of different shapes of bone nail, respectively.

FIG. 27 shows a flowchart for a method embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. To facilitate the understanding of this invention, a number of terms are defined below. Terms defined herein have meanings as commonly understood by a person of ordinary skill in the areas relevant to the present Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Ankle fusion is currently done by screws, plus or minus, plates or external fixator. Both of these have their disadvantages. The nail system when used for nail fixation has also to include the subtalar joint. So when a nail is used to fuse an ankle (tibio-talar) joint, it will have to also fuse another joint (subtalar or the talo-calcaneal joint). This means that the ankle joint cannot be fused by a nail unless another joint is fused with it. This invention to have a nail that can fuse the ankle without the need for fusing another joint with it.

Thus, the present invention provides for a novel ankle fusion nail that overcomes the problems with existing external fixators or plates, as outlined herein below. Ankle fusion by current nails will require fusion of the subtalar joint. This invention helps to fuse the ankle joint with a nail without fusing the subtalar joint.

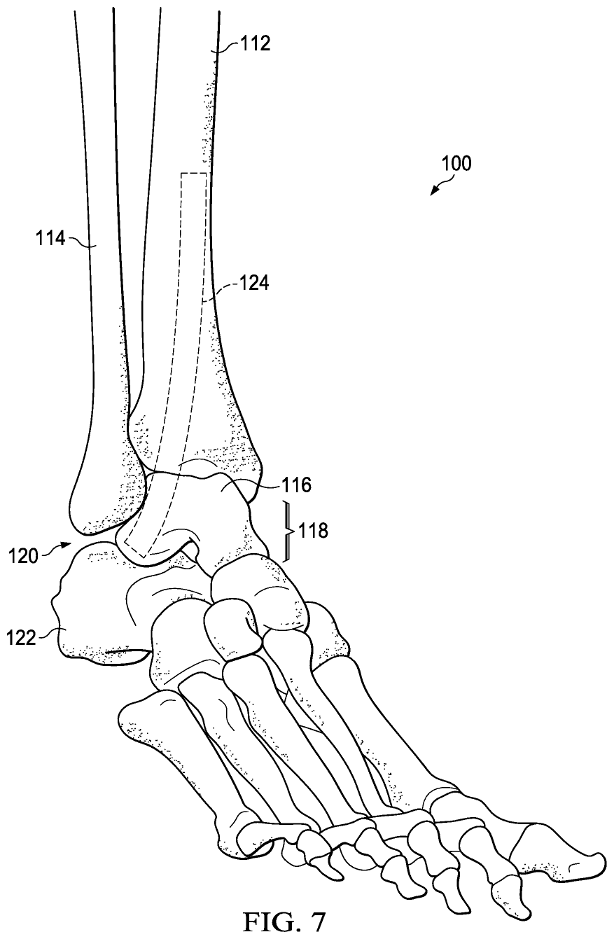

Thus, in one embodiment the present invention includes a nail that is adapted to fuse the ankle joint without crossing the subtalar joint (see FIGS. 7-11).

In certain non-limiting examples, the nail specification can include: Titanium or stainless steel, or a material of similar strength that is compatible. While not a limitation of the present invention, it may be convenient to provide the nail in a few widths, e.g., 8 mm and 10 mm (more sizes can be added). Likewise, the length can be selected from, e.g., 20-35 cm with 5 cm increments, depending on the size of the bones to be fused (e.g., pediatric versus adult). One feature of the nail is that it permits distal locking in the talus (from lateral to medial and from anterior to posterior). Another feature is that it provides for locking proximally in the tibia that is from lateral to medial. Another feature is also screws that pass from the tibia or the fibula through the nail to the talus. This will give a great amount of fixation and stability for the nail fusion construct.

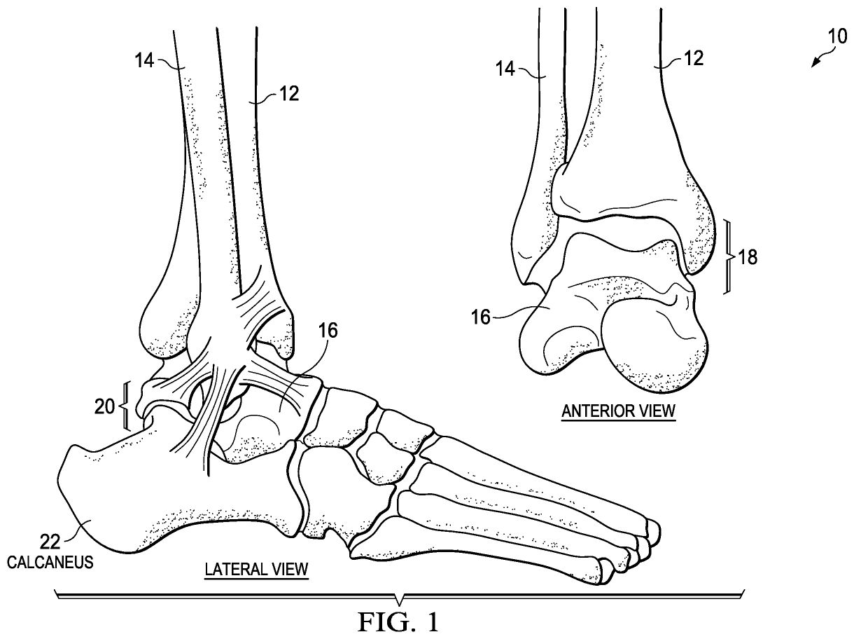

Additional novel features of the present invention are that it provides for intra medullary fusion of the ankle without crossing the subtalar joint irrespective of: (1) Nail is passing from talus to tibia or from tibia to talus; (2) Shape of nail, diameter, material; (3) Size and length; and (4) orientation and number of locking screws. FIG. 1 shows a lateral and an anterior view of the anatomy of the ankle and subtalar joint 10. Briefly, the tibia 12 is shown in relation to fibula 14. The talus 16 is shown in both views and in relation to the true ankle joint 18 and the subtalar joint 20. Finally, in the lateral view, the calcaneus 22 is also depicted.

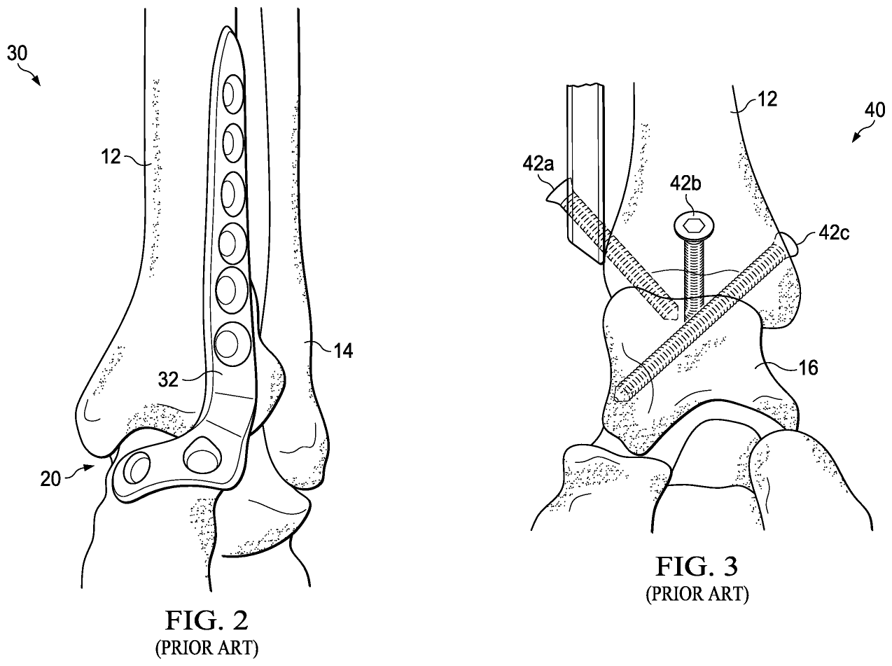

FIG. 2 shows a posterior view of an ankle fusion 30 using a plate 32 of the prior art. In this posterior view, the plate 32 is depicted connecting the tibia 12 across the ankle joint 18.

FIG. 3 shows an anterior view of an ankle fusion 40 by screws 42a, 42b and 42c depicted diagonally across the tibia 12 into the talus 16 of the prior art.

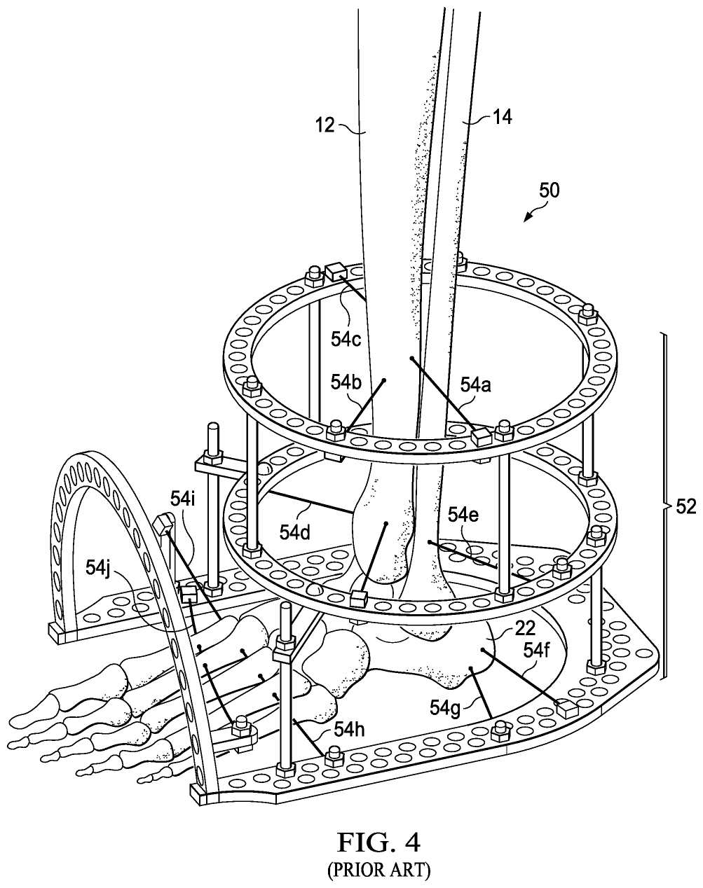

FIG. 4 shows an isometric view of an ankle fusion 50 by external fixator 52 of the prior art, in which pins 54a-j are shown connecting to the tibia 12, fibula 14, the calcaneus 22.

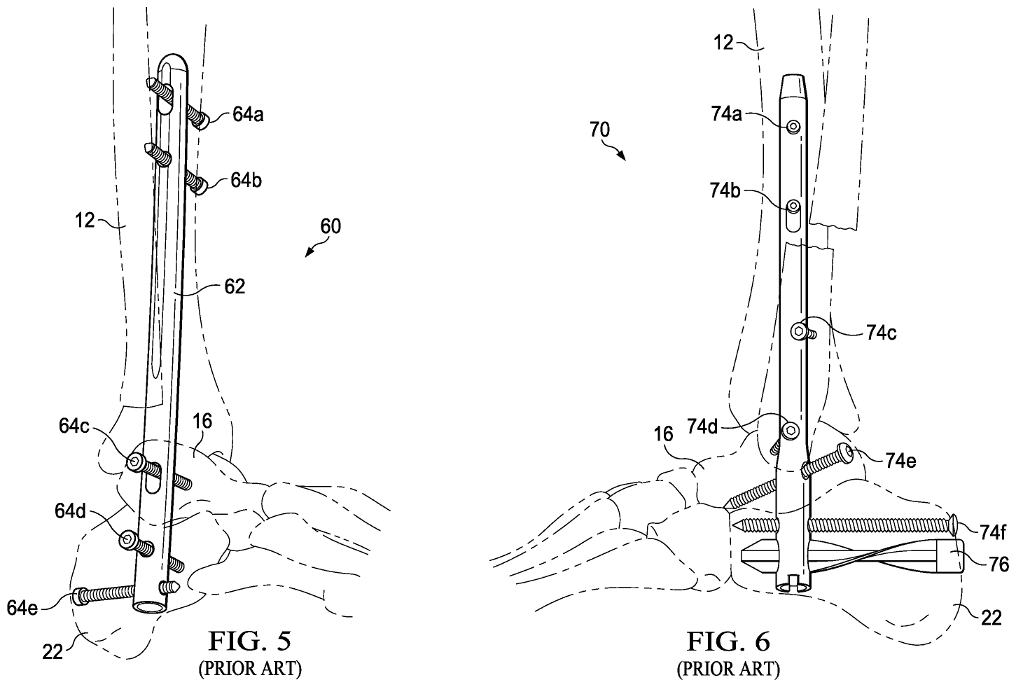

FIG. 5 shows a lateral view of an ankle fusion 60 by nail of the prior art. A straight nail 62 that is positioned inside the tibia 12 is depicted into which screws 64a-e are shown screwed into the tibia 12, talus 16 and calcaneus 22.

FIG. 6 shows a lateral and a posterior view of an ankle fusion 70 by nail of the prior art. In this version, the straight nail 72 is also depicted with various screws 74a-f and peg 76 connecting the tibia 12, the talus 16 and the calcaneus 22. In this version, the screws 74a-e are connected to nail 72, while screw 74f is screwed into the nail 72, adjacent peg 76, both of which are screwed through the calcaneus 22. Screw 74e is screwed through the subtalar joint 20.

FIG. 7 shows an isometric anterior view of an ankle fusion 100 of the present invention depicting the tibia 112 and the talus 116. Briefly, the tibia 112 is shown in relation to fibula The talus 116 is shown in both views and in relation to the true ankle joint 118 and the subtalar joint 120. Finally, in the lateral view, the calcaneus 122 is also depicted. The bone nail 124 of the present invention is shown traversing the tibia 112 and connecting into the talus 116, in a manner that does not traverse the subtalar joint 120. By providing a direct connection between the tibia 112 and the talus 116, the bone nail 124 can be used alone, without the need for screws. In some embodiments (not depicted), additional mechanical support can be provided between the bones and the nail, with support found on either the nail or the bone. This will be achieved by using locking screws in both the tibia and the talus. Compression across the fusion can be obtained by having screws that pass from the tibia or fibula on one side and the talus on the other side passing though the plate and giving extra strength to the fusion construct. The curvature of the bone nail 124 permits the nail to traverse the tibia 112 and the talus 116. Because the bone nail 124 is curved, such that it traverses the talus and enters the tibia at an angle, the bone nail 124 enters at an angle that is from lateral to medial. As such, the bone nail 124 is configured to provide for intra medullary fusion of the ankle without obstructing the subtalar joint 120.

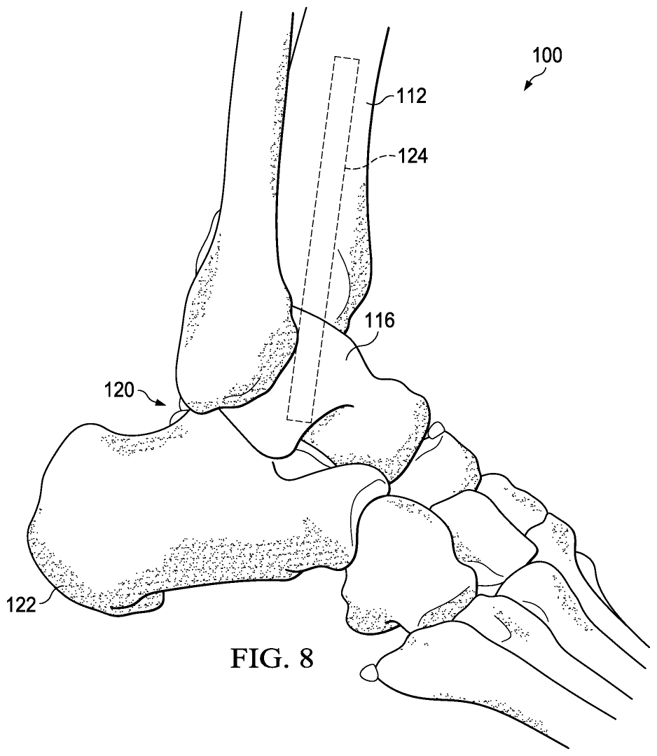

FIG. 8 shows a close-up lateral view of the bone nail 124 of the present invention positioned showing the tibia 112 and the talus 116, which in the lateral view shows that in this angle the bone nail 124 is generally straight and does not obstruct the subtalar joint 120.

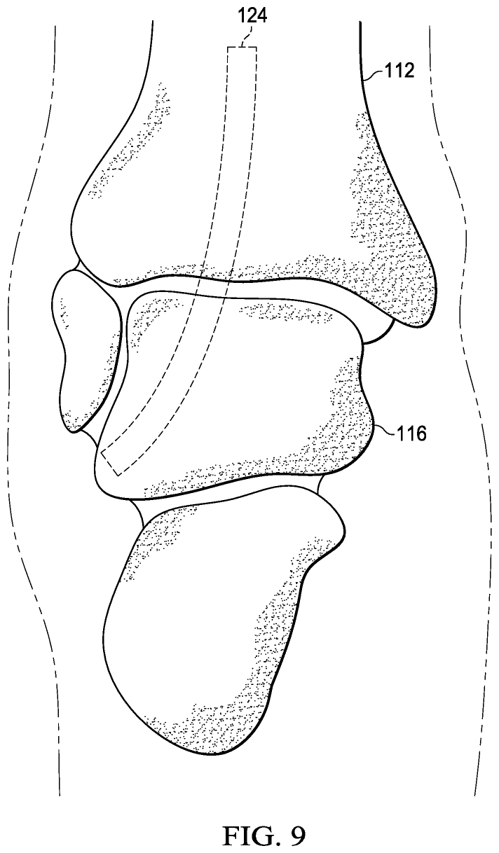

FIG. 9 shows an anterior view of the bone nail 124 of the present invention positioned showing the tibia 112 and the talus 116.

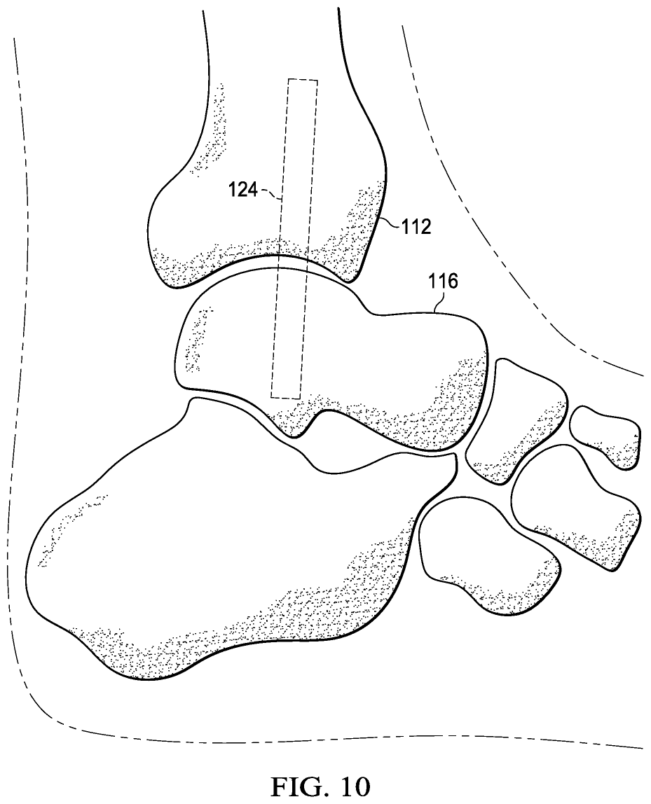

FIG. 10 shows a close-up lateral view of the bone nail 124 of the present invention positioned showing the tibia 112 and the talus 116.

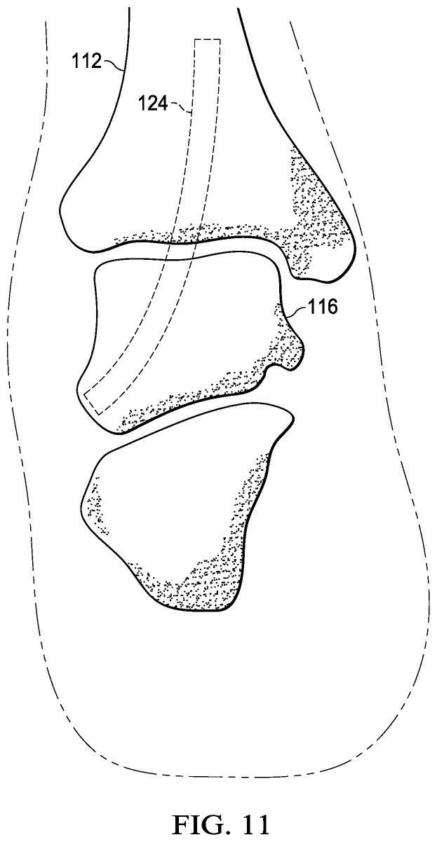

FIG. 11 shows a coronal view of the bone nail 124 of the present invention positioned showing the tibia 112 and the talus 116.

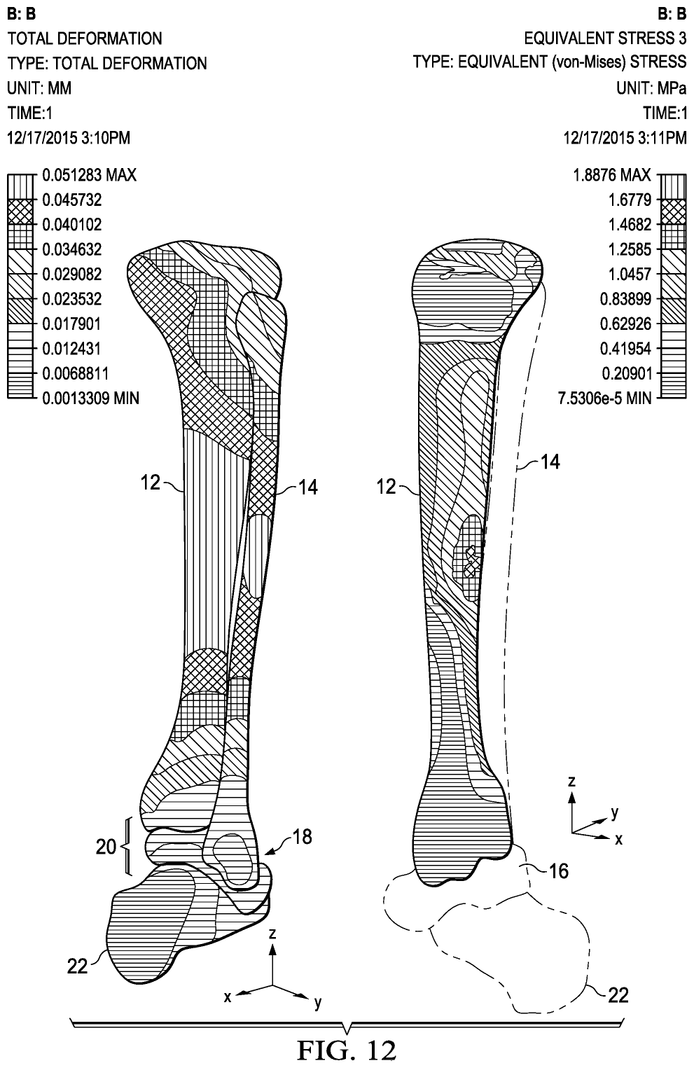

FIG. 12 shows a computational analysis was performed applying an axial load of 350N at the top area of the tibia simulating the weight bearing of an average person. Briefly, the tibia 12 is shown in relation to fibula 14. The talus 16 is shown in both views and in relation to the true ankle joint 18 and the subtalar joint 20. Finally, in the lateral view, the calcaneus 22 is also depicted. The inventors determined which the main stressed areas are in the bone and to define the most stressed sections of the nail. For this study, the nail is curved since it is expected to be the worst case if compare to straight segments carrying load, however the nail is lacking of details intentionally, it is not the final design and does not include screw holes yet, reducing the complexity of the model. For the tibia and fibula, the main and higher stresses are located close to the middle section of the bone when applying the axial force.

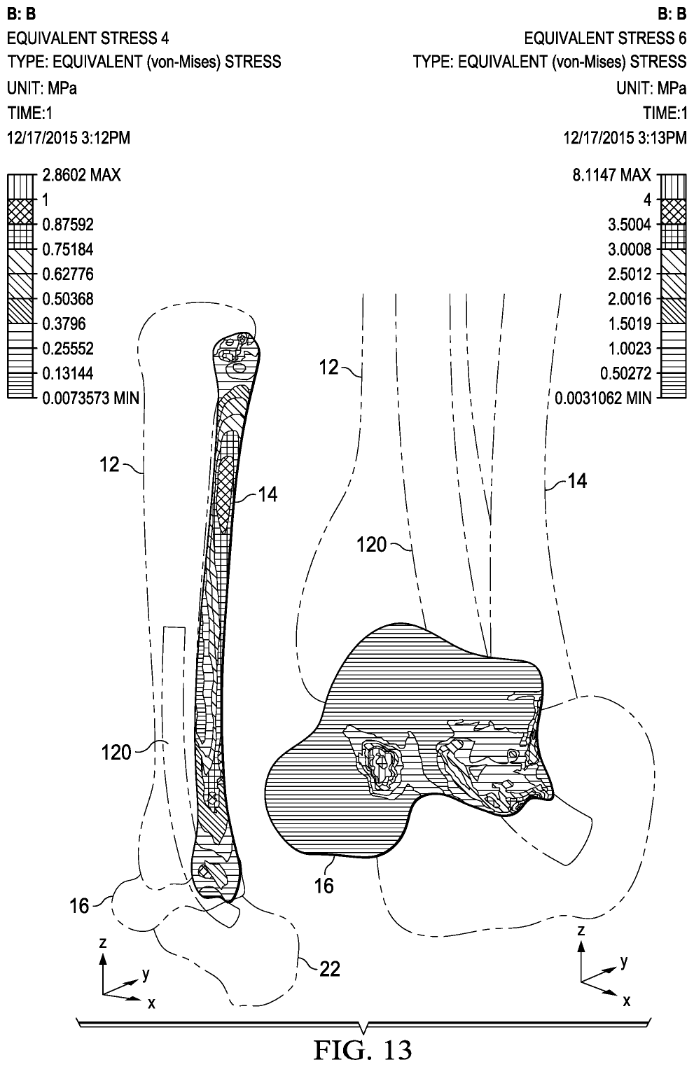

FIG. 13 shows a computational analysis in which the talus is subjected to high stress in the section around the nail insertion. The tibia 12 is shown in relation to fibula 14 and the curved nail 120. The talus 16 is shown in both views and in relation to the true ankle joint 18 and the subtalar joint 20. Finally, in the lateral view, the calcaneus 22 is also depicted. The talus 16 is subjected to high stress in the section around the curved nail 120 insertion as expected. The calcaneus 22 shows stress concentration in the areas closer to the nail 120 since the curvature of the metal transfers the stresses to any area nearby.

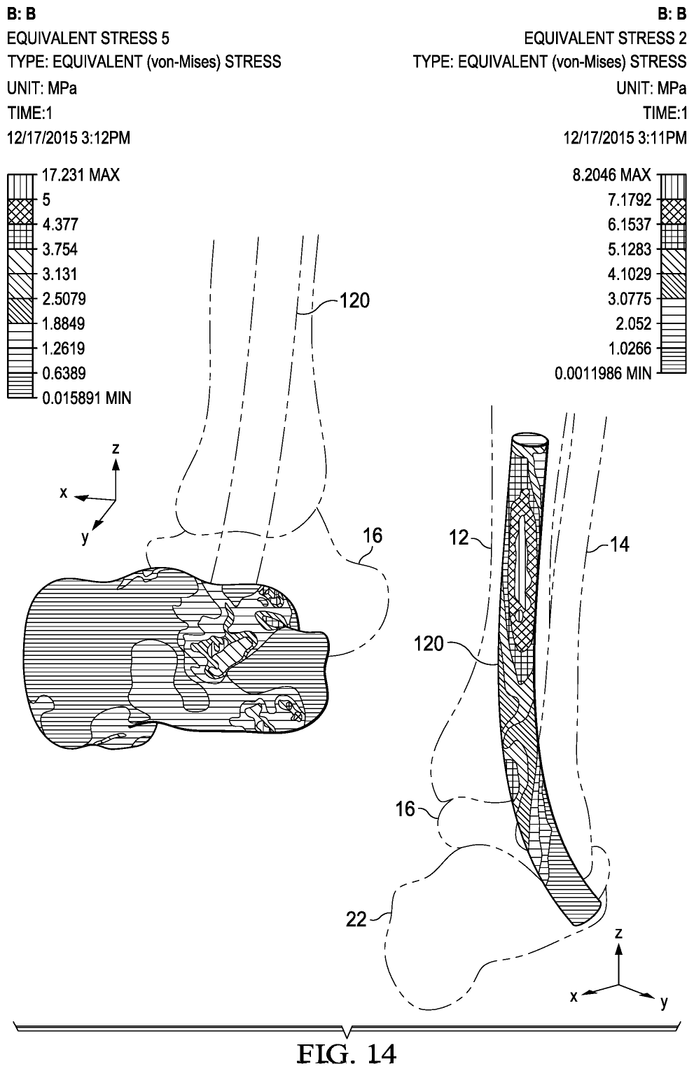

FIG. 14 shows a computational analysis in which the curved nail 120, tibia 12, and talus 16 are shown and in which the stress is concentrated in the upper section which carries the axial load at the first instance, it is expected that the addition of screws (not depicted) help to distribute the stresses in nail.

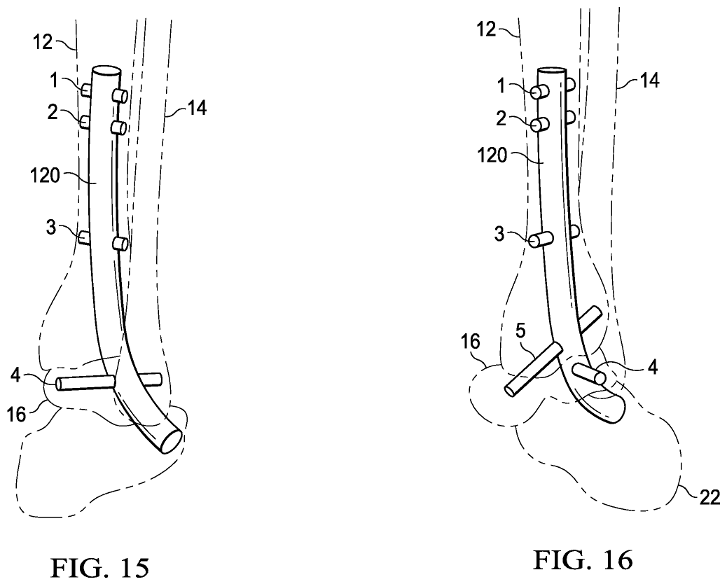

FIG. 15 shows the fixation of the nail to the bones with screws that ensures proper fixation and preventing movement during joint fusion and supporting and distributing stresses uniformly throughout the structure. The tibia 12 is shown in relation to fibula 14 and the curved nail 120. FIG. 15 shows screws 1, 2, 3, 4, in relation to the curved nail 120 of the present invention. The fixation of the nail to the bones with screws is very important and will perform different tasks such as ensuring proper fixation and preventing movement during joint fusion and supporting and distributing stresses uniformly throughout the structure. At least two screws are required to fix the nail to the tibia in the proximal section (1 & 2); the screws can be inserted from lateral to medial. In the distal section of the tibia another screw is required from medial to lateral (3). The talus can be fixed at least with a long screw inserted from posterior to anterior (4). In addition, a fifth inclined screw is recommended; it can be positioned from the tibia to the talus to increase fixation properties. In one non-limiting embodiment, with respect to the horizontal axis, the screws 1, 2 and 3 have no angle of inclination.

FIG. 16 shows the same image as in FIG. 15, but also illustrates screw 5. The tibia 12 is shown in relation to fibula 14 and the curved nail 120. Screw 5 is inserted in an inclined configuration, which is positioned from the tibia 12 to the talus 16 to increase fixation.

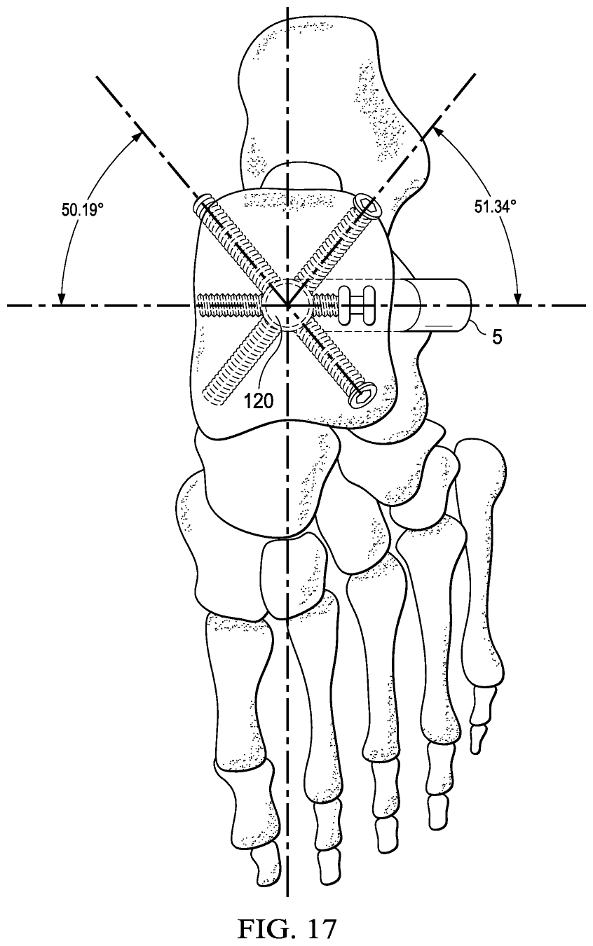

FIG. 17 a top view down the length of the curved screw 120 shows the screw 4 of the talus (4) and the screw 5 which crosses from tibia to talus (5) and the approximate angle of inclination.

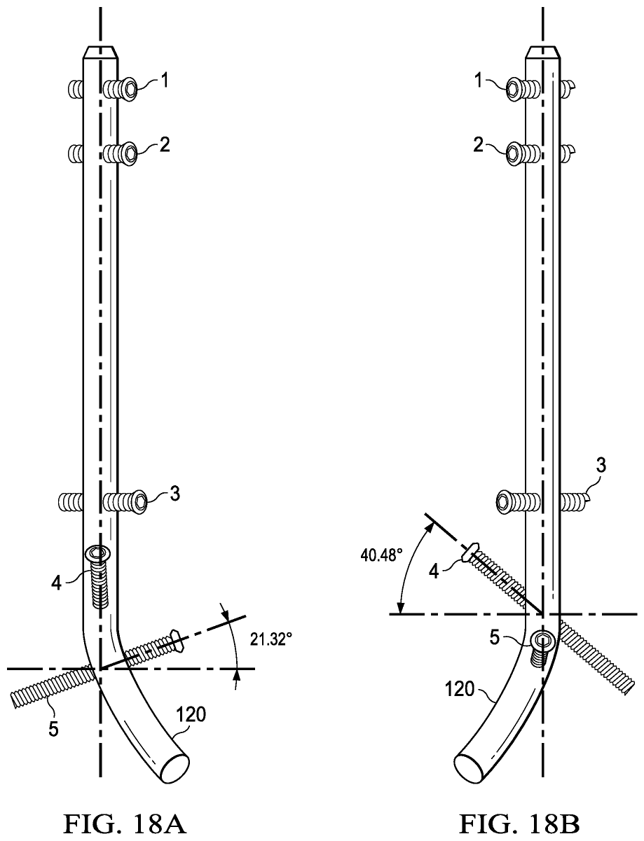

FIGS. 18A and 18B illustrate the angle of inclination for the curved nail 120, with talus screws 1-4 and for tibia/talus screw 5. Generally, the screws 1-5 are headed cortical-type screws with a screw diameter of 5.0 mm for all the bolts. Start-shape or hexagonal are good options for the screw head. One non-limiting material for the nail and the screws is a titanium alloy that has demonstrated lower mechanical failure rates and improved biocompatibility compare to stainless steel. The alloy Ti6AL4V ELI is one such option.

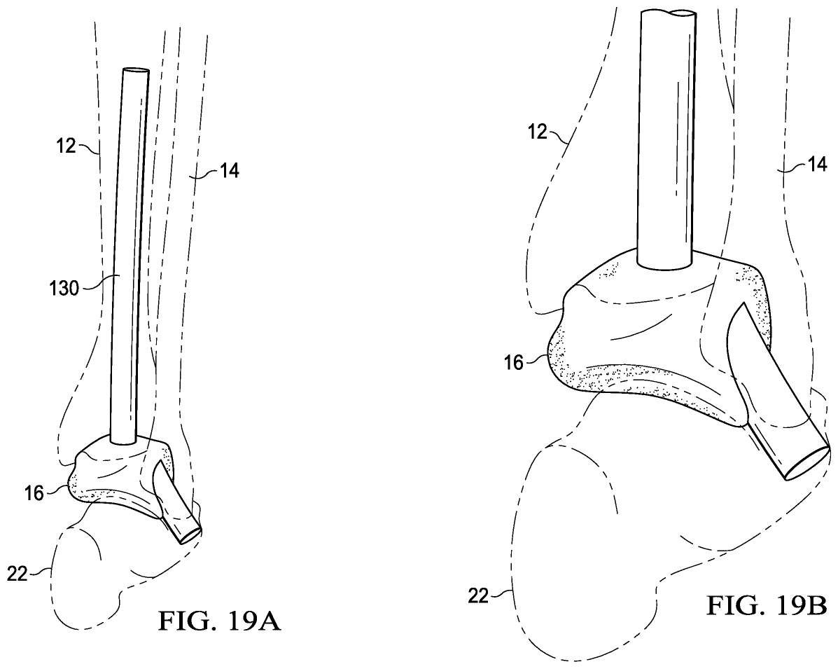

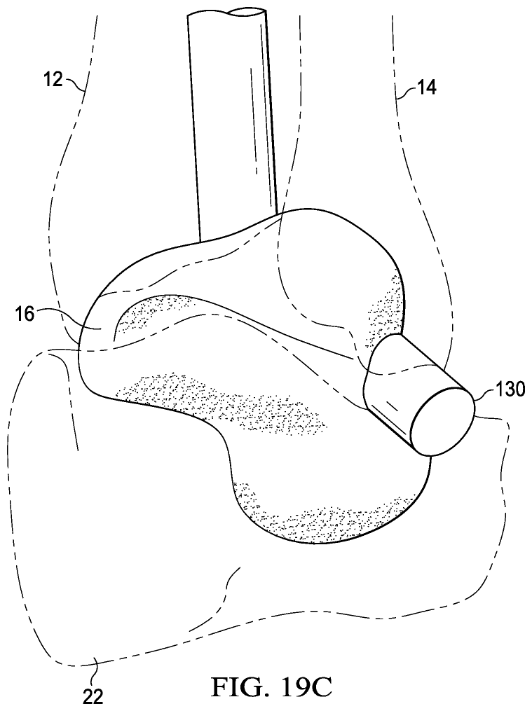

FIGS. 19A and 19B illustrate one option for the nail 130 of the present invention in which two straight segments connected each other; they have an angle of 37.24°. The tibia 12 is shown in relation to fibula 14 and the straight nail 130. The talus 16 is shown in both views in relation to the calcaneus 22, the tibia 12 and the fibula 14. This nail is called “straight nail” 130 and it is inserted from the talus 16 to the tibia 12.

FIG. 19C shows that the subtalar joint is not affected when the nail is inserted and positioned. The image below shows the clearance between the nail and calcaneus or between the nail and subtalar joint. In this configuration, the nail is not curved and the straight segments force an incline of the talus with respect to the tibia an angle of 37.24° when the nail is being inserted. After the insertion is completed, the talus and the tibia can sustained their normal position without any issues, actually the straight segments of the nail help to avoid movement between the two bones in one axis making easier the job of the securing screws. A curvature in the nail talus segment helps to have a deep insertion into the talus bone, so the designed can also be modified to have a curvature in the talus section but still a straight segment in the tibia.

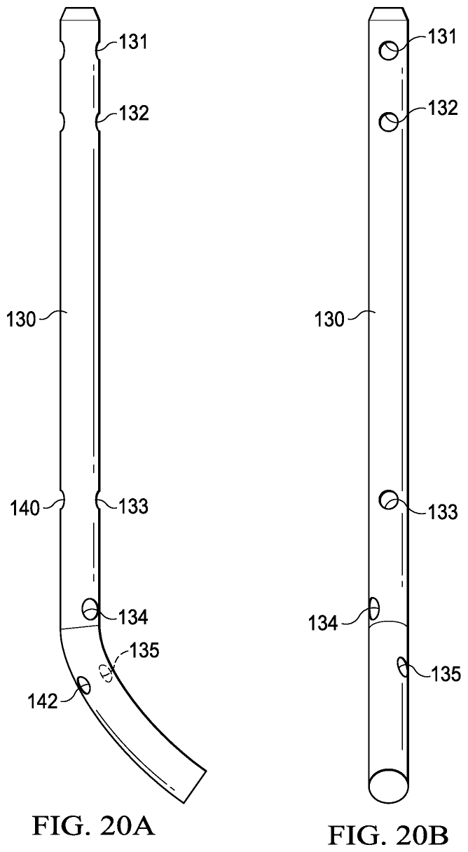

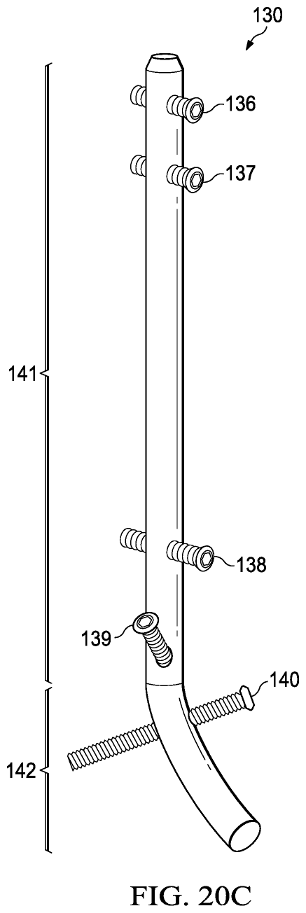

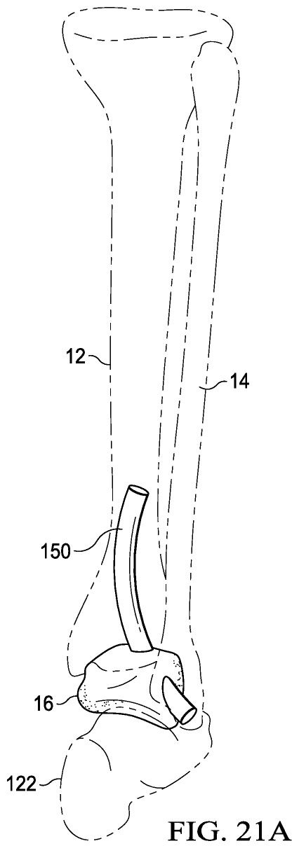

FIGS. 20A and 20B are a lateral and front view of the nail 130 and screw holes 131, 132, 133, 134 and 135. FIG. 20C is an isometric view of the nail 130, screws 136, 137, 138, 139, 140, in screw holes 131, 132, 133, 134 and 135. In certain non-limiting examples, the nail has one or more of the following features: Angle=37.24° to 45°, Radius of curvature R=7.72 cm, Nail length=22 cm (longer or shorter versions are possible in this design), Nail diameter=10 mm (8 mm version is also feasible), 5 screws: 5.0 mm diameter, 3 screws for the tibia, 1 for tibia/talus & 1 for the talus, and/or a talus rotation with respect to tibia is required) (37.24°) when inserted the straight segment of the nail. The nail 130 has an upper portion that is a straight portion 141 and a lower portion that is a curved portion 142, which can be made in a single piece, or in two pieces, although certain advantages can be found in the screw being made from a single piece, that is, of unitary construction.

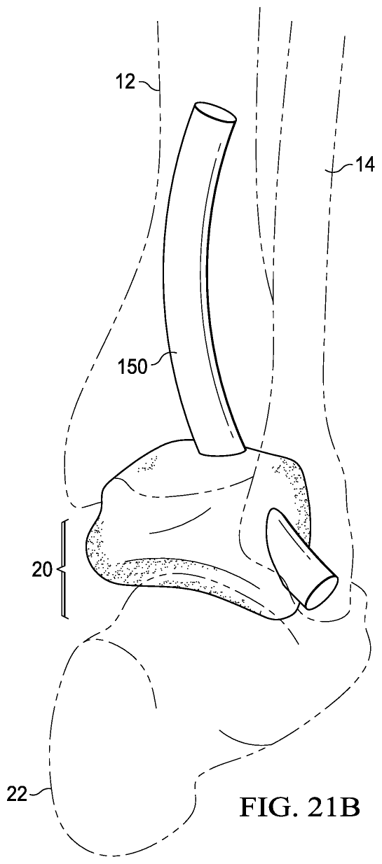

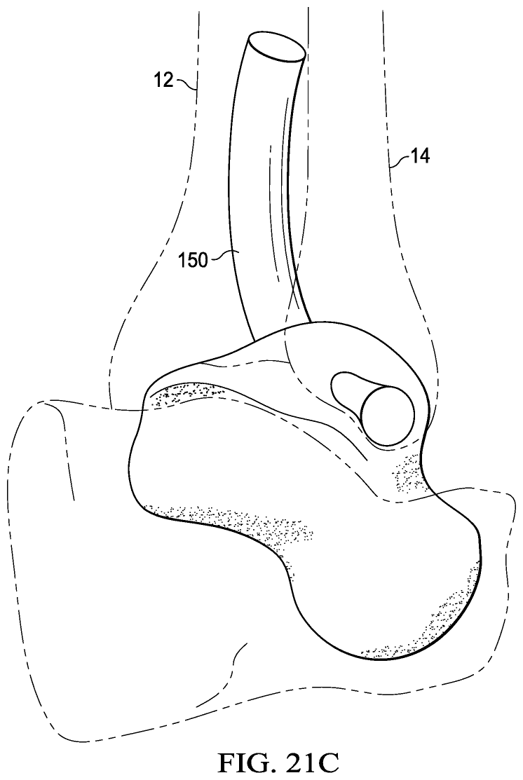

FIGS. 21A and 21B illustrate another option for the constant curved nail 150 of the present invention and shows the clearance between the constant curved nail 150 and calcaneus 22 or between the nail 150 and subtalar joint 20, and is based on constant curved nail 150 geometry. The tibia 12 is shown in relation to fibula 14 and the straight nail 130. The talus 16 is shown in both views in relation to the calcaneus 22, the tibia 12 and the fibula 14. Only one curvature is designed and will pass through the tibia 12 and the talus 16. The constant curve nail 150 has a predominately single curvature.

FIG. 21C shows the clearance between the constant curved nail 150 trajectory and the subtalar joint 20, which prevents damage in the subtalar joint 20. In this embodiment, the constant curved nail 150 is not as close to the calcaneus 22 as in the figures above, however, the constant curved nail 150 is closer to the sidewall of the tibia. In terms of insertion of the nail into the bone, the advantage of this option is important because there is no need to move the talus with respect to the tibia when insert it. However, is it important to considerer that a curved hole is needed in talus section; if the bone drill is not curved then the advantage mentioned above may not be that important.

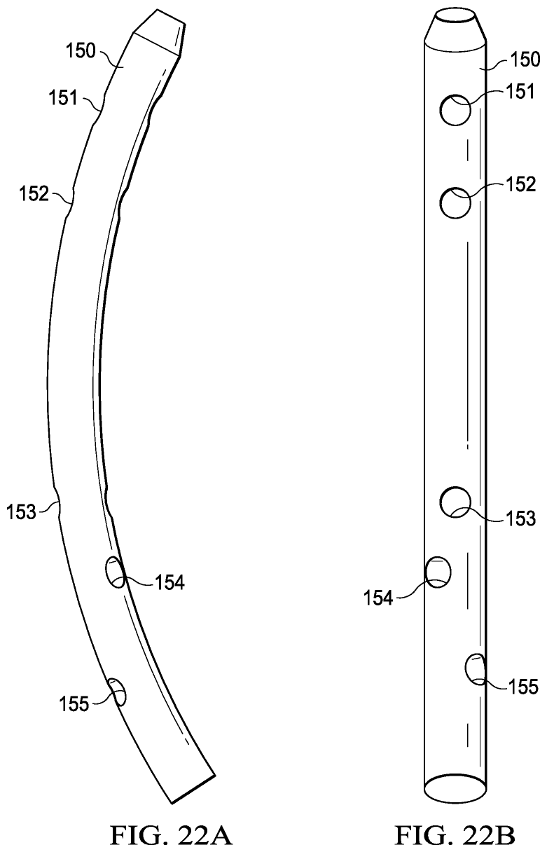

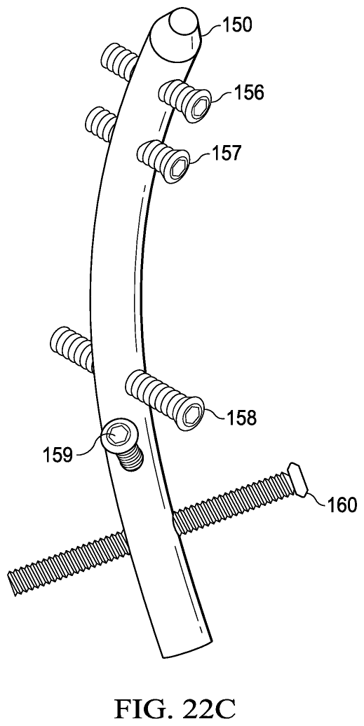

FIGS. 22A and 22B are a lateral and front view of the curved nail 150 and screw holes 151, 152, 153, 154 and 155. FIG. 22C is an isometric view of the curved nail 150, screws 156, 157, 158, 159, 160, in screw holes 151, 152, 153, 154 and 155. In certain non-limiting examples, the nail has one or more of the following features: Angle=16.58°, Radius of curvature R=12.79 cm, Nail length=13 cm (shorter version is feasible but in some cases it may be difficult to increase length in this design), Nail diameter=10 mm (8 mm version is also feasible), 5 screws: 5.0 mm diameter, 3 screws for the tibia, 1 for tibia/talus & 1 for the talus.

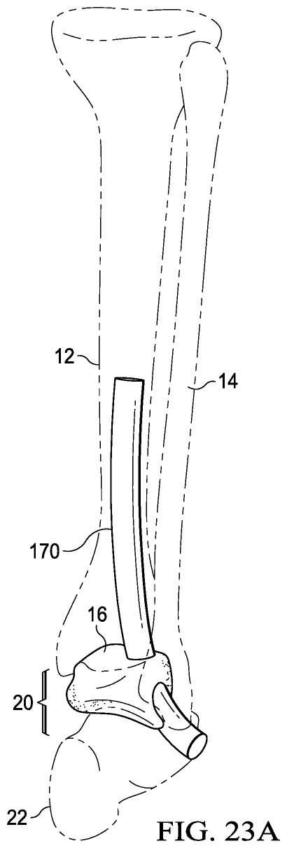

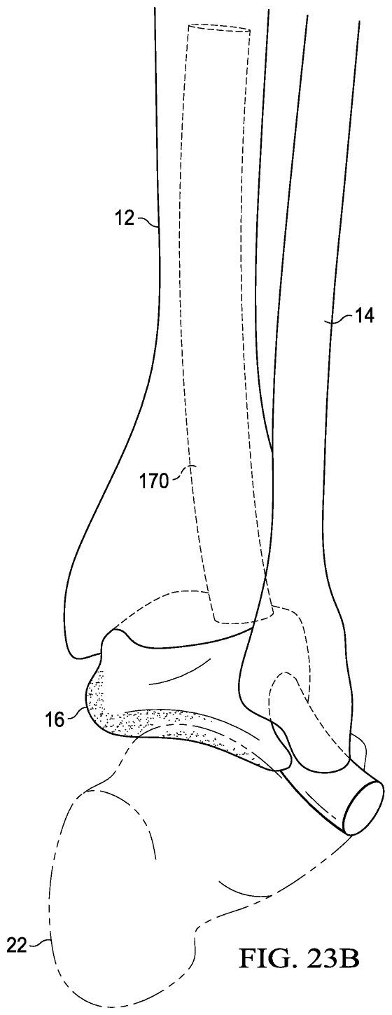

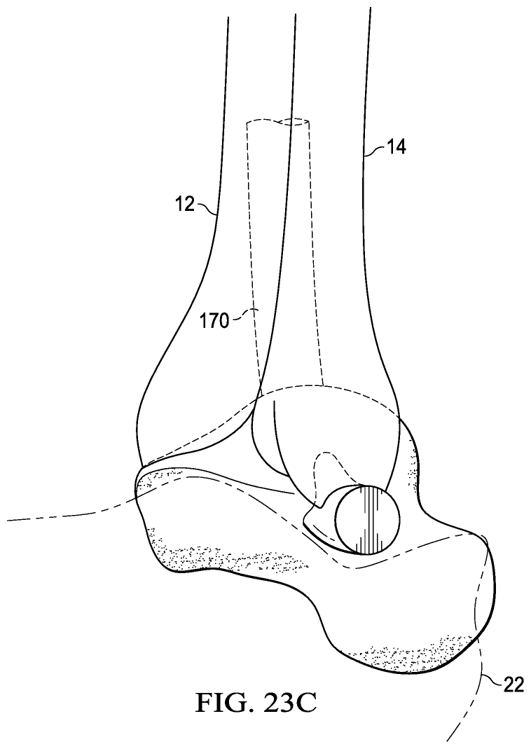

FIGS. 23A and 23B illustrate another option for the nail 170 of the present invention in which the curved nail has two different curves to form the nail 170. A first curvature is designed for the tibia segment (or upper segment) and a second more pronounce curvature is selected for the talus segment (or lower portion).

FIG. 23C shows the clearance between the nail 170 and subtalar joint is small but still enough to avoid damage in the calcaneus zone 22. This nail has the advantage of the curvature and very small movement is needed between the talus and the tibia when the nail is being implanted

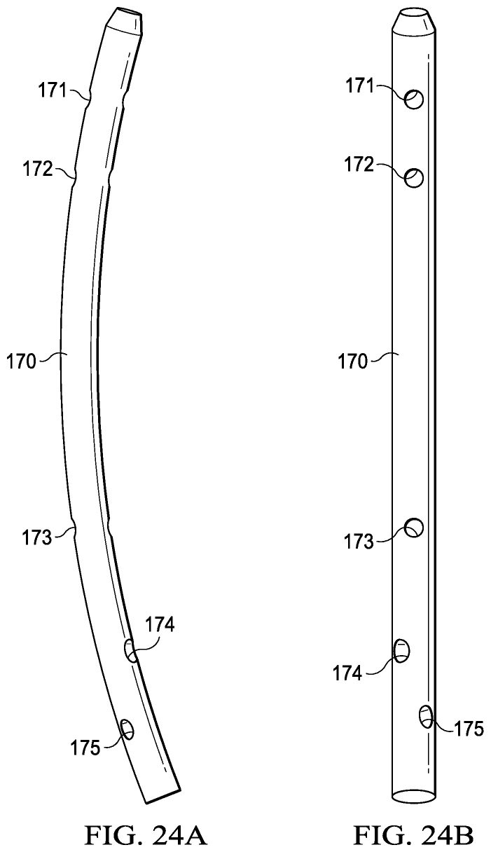

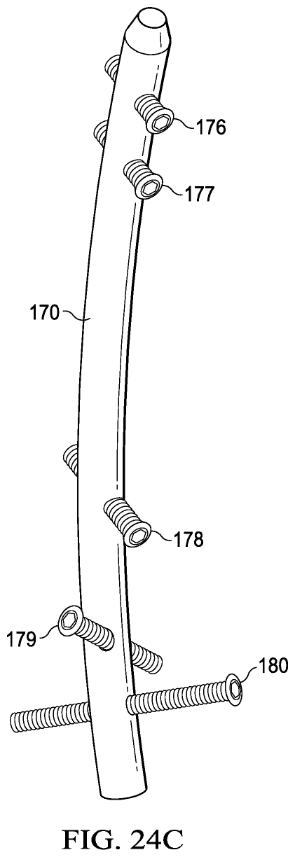

FIGS. 24A and 24B are a lateral and front view of the nail 170 and screw holes 171, 172, 173, 174 and 175. FIG. 24C is an isometric view of the nail 10, screws 176, 177, 178, 179, 180, in screw holes 171, 172, 173, 174 and 175. In certain non-limiting examples, the nail has one or more of the following features: Angle=10.99°, Radius of curvature R=32.07 cm, Nail length=20 cm (shorter version is feasible but difficult to increase length in this design), Nail diameter=10 mm (8 mm version is also feasible), 5 screws: 5.0 mm diameter, 3 screws for the tibia, 1 for tibia/talus & 1 for the talus.

The present invention further defines the position of the screw in a way to use the fibular bone to obtain more compression. This compression (if successfully implanted) makes this fusion device a much better mechanical device than other methods of fixation. By obtaining better mechanical properties of the device, this ankle fusion device is a better ankle fusion solution from both the biology and mechanics.

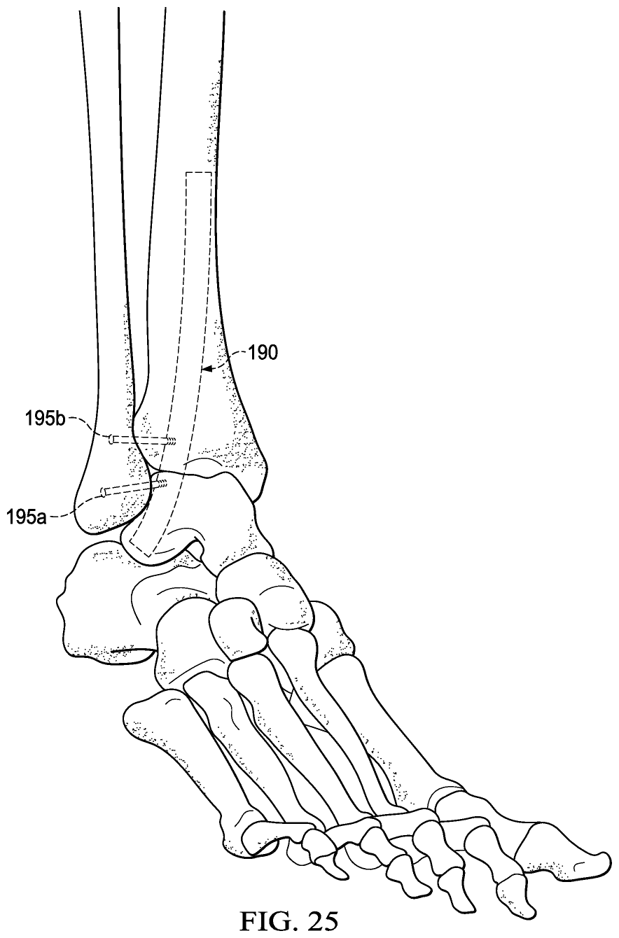

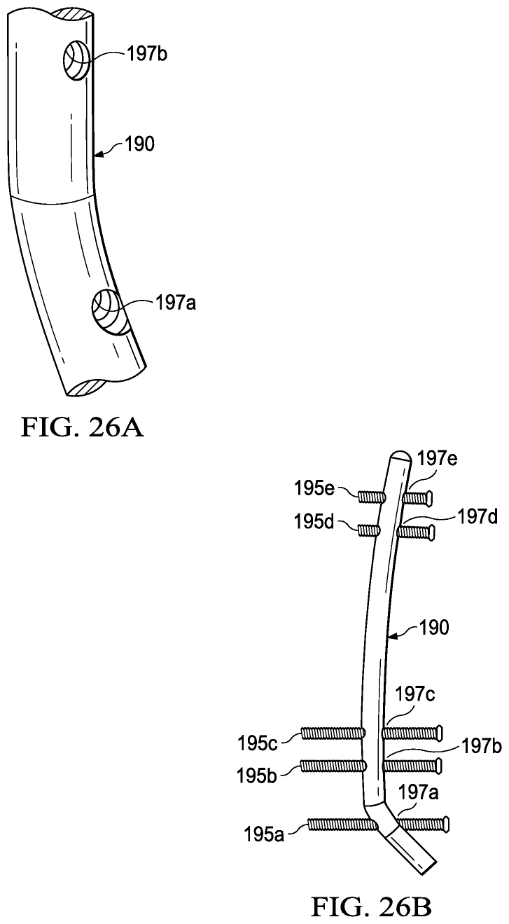

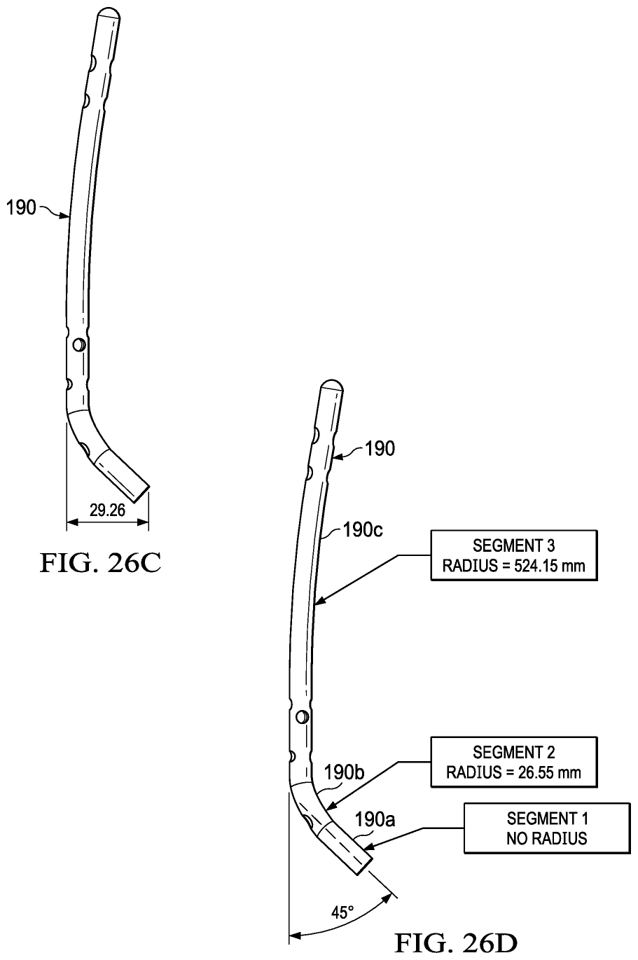

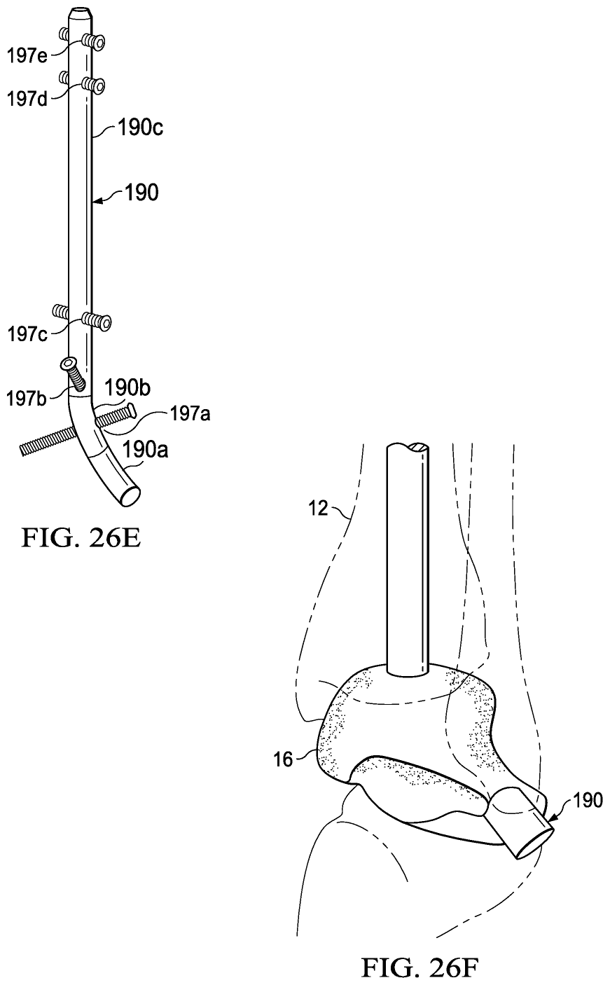

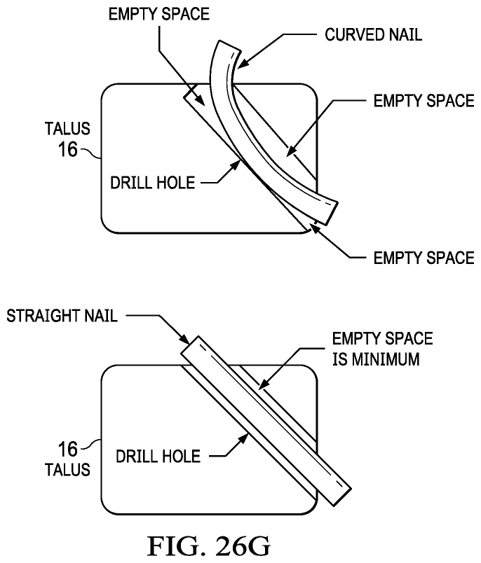

FIG. 25 shows an embodiment of the present invention in which a tibio-talar device for providing stabilizing support between a tibia 12 and a talus 16 or an ankle arthrodesis nail kit includes a bone nail 190 and one or more screws 195a, 195b. The bone nail 190 is angled from lateral to medial and anterior to posterior and is configured to provide for intra medullary fusion of the ankle without obstructing the subtalar joint. The bone nail has 2, 3, 4, 5, 6, 7, or 8 openings (not shown) that are each capable of supporting a screw 195. One or more of the openings may be threaded to engage a screw, and one of more of the openings may be capable of engaging a locking screw using a surface or a locking mechanism. The openings are positioned along the length of the bone nail 190 and are all horizontal (see FIGS. 26A-26G). At least a portion of each screw 195 proximate to the head of the screw 195 is not threaded, while at least a portion of the distal end is threaded for engagement with the bone nail 190. A similar function can be achieved if the entire length screw 195 is threaded, but two drill bits with different sizes are used to insert the screw 195. The drill bit used for the fibula should have the same diameter of the screw 195 so that the threaded portion of the screw 195 does not engage the fibula and a small drill bit is used to drill the talus or the tibia or that the screw 195 does engage the talus or the tibia. Each of the one or more screws 195 may be adapted to lock into one of the openings in the bone nail 190. A screw 195 may be inserted through a hole made in a fibula 14 and then in a hole in a talus 16 or the tibia 12 to reach a bone nail 190 that has been inserted into the tibia 12 or talus 16. The screw 195 may then engage with or be locked into the bone nail 190. The screw 195 may be used in this way to provide additional mechanical stability and compression. The nail 190 has screw holes that allows the nail to be engaged into it. This engagement of the screws in the nail 190 will allow to compress the fibula 14 to the talus 16 and tibia 12. Two such screws 195 may provide sufficient additional mechanical stability and compression, but the present invention is not limited to two screws 195. These screws 195 can either be partially threaded (a portion is threaded and a portion is not threaded) with the threaded part engaging in the bone nail 190 to gain compression) or the screws 195 can be inserted using two drill bit sizes with a bigger drill bit used to drill the fibula. This improved design of the nail 190 is driven by the need to increase fixation for the entire assembly (nail, screws and bones), therefore allows for some compression in the ankle joint, resulting in proper tibia and talus fusion. For that purpose threaded holes were added so screws can be fixed to the nail in key locations with respect to tibia and talus. Talus nail hole and tibial (more distal) nail hole are now threaded and are generally horizontal when the patient is standing. Further, all the nail screw holes are now oriented horizontally to allow friendly insertion from lateral to medial with no angle

FIG. 26A depicts an embodiment of the present invention in which the two lowest openings 197a, 197b, of the bone nail 190 are threaded to engage screws 195a, 195b inserted into them to achieve more compression. Other openings among the openings in the bone nail 190 may be similarly threaded to engage screws 195 inserted into them.

FIG. 26B depicts an embodiment of the present invention in which five lowest openings 197a, 197b, 197c, 197d, 197e of the bone nail 190, here depicted with screws 195a, 195b, 195c, 195d, 195e inserted in them, respectively, are oriented horizontally to permit easier access for the insertion of screws 195 into them. Other openings among the openings in the bone nail 190 may be similarly oriented horizontally to permit easier access for the insertion of screws 195 into them. Among the screws that are usable in this embodiment are 4.0HA cortical screws.

FIG. 26C depicts an embodiment of the present invention in which a non-limiting, exemplary horizontal width of the bone nail 190 is 29.26 mm. FIG. 26D depicts an embodiment of the present invention in which the lowest segment 190a of three segments is a straight segment with no radius, the middle segment 190b has a radius of 26.55 mm and the upper segment 190c has a radius of 524.15 mm. The curvature of the middle segment is therefore more pronounced. This design of bone nail 190 can facilitate insertion and in the same time decrease the amount of bone needed to be removed during reaming before nail insertion.

FIG. 26E shows another embodiment of the present invention, the bone nail 190 with lowest segment 190a, middle segment 190b, and upper segment 190c, and holes 197a, 197b, 197c, 197d, and 197e. FIG. 26F shows the bone nail 190 inserted in the tibia 12 and the talus 16.

FIG. 26G shows the amount of bone that needs to be removed with curved designs of bone nail 190 and with a straight lower design of bone nail 190. The curved design requires more bone to be removed from the talus than the straight design. Also, a shorter-length bone nail 190 of the straight design can be used. A longer-length bone nail 190 of the curved design cannot be used as it will hit the cortex of the tibia, causing a fracture.

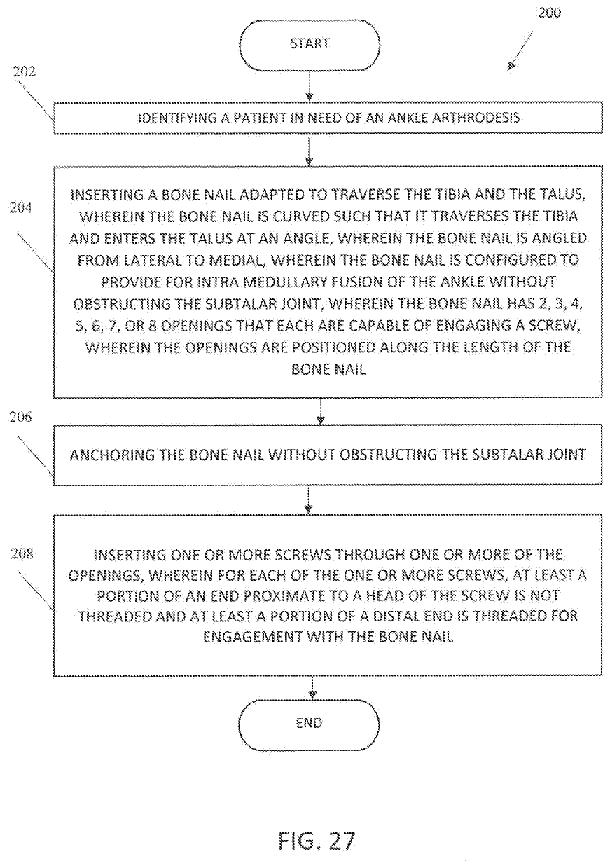

FIG. 27 shows a method embodiment of the present invention, a method 200 for conducting an ankle arthrodesis system for providing stabilizing support between a tibia and a talus may include block 202, identifying a patient in need of an ankle arthrodesis. Method 200 may also include block 204, inserting a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a screw, wherein the openings are positioned along the length of the bone nail. Method 200 may further include block 206, anchoring the bone nail without obstructing the subtalar joint. Method 200 may further include block 208, inserting one or more screws through one or more of the openings, wherein for each of the one or more screws, at least a portion of an end proximate to a head of the screw is not threaded and at least a portion of a distal end is threaded for engagement with the bone nail, and wherein each of the one or more screws is adapted to lock into one of the openings in the bone nail.

The embodiments depicted in FIGS. 25, 26A, 26B, 26C, 26D, 26E, 26F, 26G, and 27 allow an increase in fixation for the entire assembly of bone nail screws, and bones. It can allow some compression in the ankle joint, resulting in proper tibia and talus fusion. For that purpose threaded holes allow screws to be fixed to the bone nail in key locations with respect to the tibia and the talus.

Finally, the present invention is distinguishable from the ankle fusion nail apparatus and method (WO 2014062205 A1) “Curved Tibiotalar Fusion Nail And Method Of Use” for the following reasons. The design taught therein has one or more inherent problems and is not similar to the nail of the present invention due to these reasons. (1) The nail taught therein can only be of certain short length, thus, the nail taught therein cannot extend to the tibial shaft. Please refer to description of FIGS. 22A and 22B of the present invention for a detailed explanation. (2) Due to the curved design, the nail taught therein can not be reamed in the talus or the tibia (tibial reaming is nearly impossible, talus reaming will result in major bone loss and possible fracture of the talus). Please refer to FIG. 19 of the present invention and the description of that figure. (3) The nail taught therein is inserted medially which is technically difficult/nearly impossible. Major nerve (posterior tibial nerve) and artery (posterior tibial are present in this area. Insertion can cause severe injury to the nerve and artery. Also tibialis posterior tendon can be injured causing flat foot deformity. The nail of the present invention is designed to be inserted from the lateral side, which is a much safer approach. (4) The locking screws of the present invention are designed in a manner that makes the device extremely stable and achieves a quicker union (refer to FIGS. 25 and 26 and description of the present invention of these two figures for more details). (5) A comparison of the design of the present invention with the curved designs and the present inventors found that the design was better bio-mechanically, namely, the CC1 (curved) design provides the advantage of not needing to angulate the talus during insertion; however, a curved hole is required for insertion, and thus a curved reamer would be necessary which obviates its main advantage. It is also limited in the inability to increase the length of the nail for the purposes of increased stability as the curvature of the nail abuts the tibia at a relatively short distance of 13 cm with a 12.79 cm radius of curvature at an angle of 16.58 degrees. This conclusion after biomechanical studies was due to the increased talar bone loss required for insertion and the associated decreased mechanical stability this causes, this design was deemed inadequate.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCE

“A New Technique for Obtaining Bone Graft in Cases of Distal Femur Nonunion: Passing a Reamer/Irrigator/Aspirator Retrograde Through the Nonunion Site.” Eisenstein E D, Waterman B R, Kanlic E M, Abdelgawad A A. Am J Orthop (Belle Mead N.J.). 2016 November/December; 45(7):E493-E496.

1. A tibio-talar device for providing stabilizing support between a tibia and a talus comprising:
a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a screw, wherein the openings are positioned along the length of the bone nail, and wherein the screw engages the bone nail to lock its rotation in relation to the tibia and talus and optionally for compression of two or more bones selected from tibia to talus, fibula to tibia, or fibula to talus.

2. The device of claim 1, further comprising one or more screws, wherein at least of the one or more screws is adapted to engage one of the openings in the bone nail.

3. The device of claim 1, wherein the bone nail is at least one of: further affixed to the tibia and talus with a biocompatible adhesive; contoured to lock proximally in the tibia from lateral to medial; provided in increments of approximately 5 cm; or is at least one of titanium, stainless steel, nitinol or other biocompatible material.

4. The device of claim 1, further comprising one or more openings to receive screws that pass from the tibia or the fibula through the bone nail to the talus, or wherein at least one of the openings is threaded to engage the at least one of the one or more screws to compress the fibula to both the tibia and talus to enhance fusion, or the openings have a longitudinal axis from lateral to medial, and are horizontal.

5. The device of claim 1, wherein the bone nail has an upper portion, a middle portion, and a lower portion, wherein the upper portion and the lower portion have the same curvature, the upper portion is straight and the lower portion is curved, wherein the lower and upper portions are curved but have different curvatures, or wherein the upper portion is curved, the middle portion is curved, and the lower portion is straight.

6. (canceled)

7. The device of claim 1, wherein for at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded;
at least one of the screws is adapted to engage bone tissue with a threaded portion to enhance compression and stability of fusion;
at least one of the screws is adapted to lock into the bone nail.

8. (canceled)

9. (canceled)

10. (canceled)

11. An ankle arthrodesis nail kit comprising:
a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a locking screw using a surface or a locking mechanism, wherein the openings are positioned along the length of the bone nail, and wherein the screw engages the bone nail to lock its rotation in relation to the tibia and talus and optionally for compression of two or more bones selected from tibia to talus, fibula to tibia, or fibula to talus.

12. The kit of claim 11, further comprising one or more screws, wherein for each of the one or more screws, at least a portion of an end proximate to a head of the screw is not threaded and at least a portion of a distal end is threaded for engagement with the bone nail, and wherein each of the one or more screws is adapted to lock into one of the openings in the bone nail.

13. The kit of claim 11, wherein the bone nail is at least one of: further affixed to the tibia and talus with a biocompatible adhesive; contoured to lock proximally in the tibia from lateral to medial; provided in increments of approximately 5 cm; or is at least one of titanium, stainless steel, nitinol or other biocompatible material.

14. The kit of claim 11, further comprising one or more openings to receive screws that pass from the tibia or the fibula through the bone nail to the talus, or wherein at least one of the openings is threaded to engage the at least one of the one or more screws to compress the fibula to both the tibia and talus to enhance fusion, or the openings have a longitudinal axis from lateral to medial, and are horizontal.

15. The kit of claim 11, wherein the bone nail has an upper portion, a middle portion, and a lower portion, wherein the upper portion and the lower portion have the same curvature, the upper portion is straight and the lower portion is curved, wherein the lower and upper portions are curved but have different curvatures, or wherein the upper portion is curved, the middle portion is curved, and the lower portion is straight.

16. (canceled)

17. The kit of claim 11, wherein for at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded;
at least one of the screws is adapted to engage bone tissue with a threaded portion to enhance compression and stability of fusion;
at least one of the screws is adapted to lock into the bone nail.

18. (canceled)

19. (canceled)

20. (canceled)

21. A method for conducting an ankle arthrodesis system for providing stabilizing support between a tibia and a talus, the method comprising:
identifying a patient in need of an ankle arthrodesis;
inserting a bone nail adapted to traverse the tibia and the talus, wherein the bone nail is curved such that it traverses the tibia and enters the talus at an angle, wherein the bone nail is angled from lateral to medial, wherein the bone nail is configured to provide for intra medullary fusion of the ankle without obstructing the subtalar joint, wherein the bone nail has 2, 3, 4, 5, 6, 7, or 8 openings that each are capable of engaging a screw, wherein the openings are positioned along the length of the bone nail, and wherein the screw engages the bone nail to lock its rotation in relation to the tibia and talus and optionally for compression of two or more bones selected from tibia to talus, fibula to tibia, or fibula to talus;
anchoring the bone nail without obstructing the subtalar joint; and
inserting one or more screws through one or more of the openings, wherein for each of the one or more screws, at least a portion of an end proximate to a head of the screw is not threaded and at least a portion of a distal end is threaded for engagement with the bone nail.

22. The method of claim 21, wherein opening is defined further as being adapted for the bone anchor to at least one of traverse from talus to tibia or traverse from tibia to talus.

23. The method of claim 21, wherein the bone nail is at least one of: further affixed to the tibia and talus with a biocompatible adhesive; contoured to lock proximally in the tibia from lateral to medial; provided in increments of approximately 5 cm; or is at least one of titanium, stainless steel, nitinol or other biocompatible material.

24. The method of claim 21, wherein the bone nail comprises one or more openings to receive screws that pass from the tibia or the fibula through the bone nail to the talus.

25. The method of claim 21, wherein the bone nail has an upper portion, a middle portion, and a lower portion, wherein the upper portion and the lower portion have the same curvature, the upper portion is straight and the lower portion is curved, wherein the lower and upper portions are curved but have different curvatures, or wherein the upper portion is curved, the middle portion is curved, and the lower portion is straight.

26. The method of claim 21, wherein the bone nail has 2, 3, 4, 5, 6, or 8 openings that each are capable of supporting a screw, wherein the openings are positioned along the length of the bone nail, and wherein optionally one of the openings permits affixing the bone screw to the talus.

27. The method of claim 21, wherein at least one of the openings is threaded to engage the one or more screws to compress the fibula to both the tibia and talus to enhance fusion, or wherein at least one of the openings is threaded to engage the at least one of the one or more screws to compress the fibula to both the tibia and talus to enhance fusion, or the openings have a longitudinal axis from lateral to medial, and are horizontal.

28. The method of claim 21, wherein for at least one of the screws, at least a portion of an end proximate to a head of the at least one of the screws is not threaded and at least a portion of a distal end of the at least one of the screws is threaded for engagement with the bone nail, or substantially the entire length of the at least one of the screws is threaded
at least one of the screws is adapted to engage bone tissue with a threaded portion to enhance compression and stability of fusion;
at least one of the screws is adapted to lock into the bone nail.

29. (canceled)

30. (canceled)

31. (canceled)

32. A tibio-talar device for providing stabilizing support between a tibia and a talus comprising:
a bone nail adapted to traverse the tibia and the talus, wherein the bone nail is curved such that it traverses the tibia and enters the talus at an angle, wherein the bone nail is angled from lateral to medial and anterior to posterior, wherein the bone nail is configured to provide for intra medullary fusion of the ankle without obstructing the subtalar joint, wherein the bone nail has 2, 3, 4, 5, 6, 7, or 8 openings that each are capable of engaging a locking screw using a surface or a locking mechanism, wherein the openings are positioned along the length of the bone nail, and wherein the screw engages the bone nail to lock its rotation in relation to the tibia and talus and optionally for compression of two or more bones selected from tibia to talus, fibula to tibia, or fibula to talus; and
one or more screws, wherein for each of the one or more screws, at least a portion of an end proximate to a head of the screw is not threaded and at least a portion of a distal end is threaded for engagement with the bone nail, and wherein each of the one or more screws is adapted to lock into one of the openings in the bone nail.
